(19) United States
(12) Patent Application Publication
(10) Pub. No.: US 2008/0064060 A1
(43) Pub. Date: Mar. 13, 2008

(54) Method For Productoin Of Soluble Mhc Proteins

(76) Inventor: Rainer Blasczyk, Burgwedel (DE)

Correspondence Address: WILLIAM COLLARD;COLLARD & ROE, P.C., 1077 NORTHERN BOULEVARD, ROSLYN, NY, 11576, US

(21) Appl. No.: 11/579,705

(22) PCT Filed: Apr. 16, 2005
(86) PCT No.: PCT/EP2005/04056

§ 371 Date: Nov. 6, 2006

(30) Foreign Application Priority Data
May 6, 2004 (DE) 10 2004 022 435.8

(51) Int. Cl. C07K 16/00; C12P 21/04

(52) U.S. Cl. 435/69.1; 530/350

(57) ABSTRACT

The invention relates to a recombinant, purified MHC protein, which essentially comprises the same conformation functional activity and binding characteristics for specific antibodies and antigens as the native MHC protein. The invention also relates to a method for producing the protein. To obtain a protein, whose characteristics resemble those of the native protein in the closest possible manner, the protein is soluble and not truncated.

The invention relates to a recombinant, purified MHC protein, which has essentially the same conformation, functional activity, and binding properties for specific antibodies, peptides, T cell receptors, and NK cells and antigens as the native MHC protein.

Human leukocyte antigens, abbreviated as HLA molecules, are of central importance within adaptive immunity defense. Their function consists of presenting peptides at the cell surface, with regard to T lymphocytes. In this connection, the antigen specificity of the T cells depends on the major histocompatibility complex, abbreviated as MHC, genotype, something that is referred to as MHC restriction. This MHC-bound specificity represents the basis for the ability of the immune system to differentiate between self and non-self, e.g. infection pathogens, virally infected cells, or allotransplants. The genes of the HLA complex are localized on the short arm of chromosome 6, 6p21. For historical reasons, a differentiation is made among three MHC regions, which are designated as Class II or Class III towards the centromere, and as Class I towards the telomere. HLA Classes I and II, in particular, are characterized by enormous allelic polymorphism, which, according to the current state of knowledge, comprises 540 HLA-B alleles and 418 HLA-DRB alleles, among other things. In the case of HLA Class I molecules, these are heterodimeric glycol proteins that are composed of a heavy, variable α chain and a light, invariable β chain, β2 microglobulin. Parts of the α1 and α2 domains together form the peptide-binding region PBR. Octadecameric to undecameric fragments of endogenic proteins are bound non-covalently in this pit-like depression of the platform structure, and presented relative to CD8⁺ cytotoxic T cells. The α1 and α2 domains are characterized by marked amino acid polymorphism, in the region of the PBR, while the variability in the other molecule segments is significantly lower.

In the case of the HLA Class II, as well, these are heterodimeric molecules. These consist of an α chain and a β chain, in each instance, which differ slightly from one another in terms of molecular weight. In the case of HLA Class II, both chains are jointly involved in the formation of the platform structure with their α1 and β1 domain, respectively. In contrast to HLA Class I, protein fragments having a length of 15-24 AS and taken up by way of the exogenic antigen presentation path are presented here. HLA Class II molecules are expressed on professionally antigen-presenting cells (macrophages, dendritic cells, B lymphocytes), as well as on activated T cells. The presentation of the peptides takes place relative to CD4⁺ inflammatory T cells or T helper cells. The amino acid polymorphisms present at the α1 and α2 domains, in the case of HLA Class I molecules, and in the α1 and β1 domains, in the case of HLA Class II, establish the peptide bonding behavior of the HLA molecule, in each instance, as well as the conformation of the peptides presented.

Because of the low average family size that exists in industrialized countries, an HLA-genotypically identical sibling donor can only be made available in the case of approximately 30% of the patients who are candidates for stem cell transplantation. For this reason, blood stem cells are being transplanted from HLA-matched unrelated donors, with increasing frequency. A significant problem in the case of allogenic blood stem cell transplantation is graft-versus-host disease (GVHD), in which donor lymphocytes recognize allogenic HLA characteristics in the recipient tissue. The influence of HLA mismatches on the risk of the development of severe acute GVHD or transplant rejection (host-versus-graft reaction) was examined in a number of studies. In the predominant majority of the studies, HLA Class I mismatches, particularly for HLA-A and B, proved to be clear risk factors both in the GVH direction and in the host-versus-graft (HVG) direction. With regard to HLA Class II, mismatches at the gene sites that code DRB1 and DQB1, which code the β chains of the HLA-DR and DQ characteristics, in particular, were connected with an increased risk for the occurrence of severe acute GVHD, in the case of the predominant majority of the studies. The significance of mismatches at the DPB1 gene site is currently being discussed and is controversial. With regard to the other HLA Class II gene sites, there are currently too few data for an assessment of their transplantation relevance. Taking the current data situation into account, there is currently a consensus in Germany that in the case of allogenic blood stem cell transplantation, HLA matching of donor and recipient should include the HLA Class I characteristics HLA-A and B, i.e. low-resolution typing on the allele group level, as well as the Class II gene sites DRB1 and DQB1, i.e. high-resolution molecular gene examination on the allele level. On the cut-off date of Jul. 22, 2003, there were 8,575,256 potential stem cell donors and/or umbilical cord units available worldwide, a number that is continuously growing because of information campaigns and calls for stem cell donation. Thus, it can be assumed that in the future, more phenotypically HLA-identical unrelated donors will be available for a growing number of patients, and that it will be possible to raise the requirements with regard to donor/recipient selection, for example by increasing the examination accuracy or by means of including additional characteristics.

Nowadays, organ transplantation represents a standardized method for the treatment of organ failure. The organs that are transplanted most frequently are the heart, liver, lung, kidney, and pancreas. In 2003, approximately 3,500 kidneys, 1,100 livers, 400 pancreata, 600 hearts, and 300 lungs were transplanted within the Eurotransplant Foundation of Belgium, Germany, Luxembourg, the Netherlands, Austria, Slovenia.

According to the present state of scientific knowledge, the tissue compatibility between donor and recipient is based on the conformity between the antigens of the HLA system. For kidney and heart transplantation, the best results are achieved if the HLA antigens of donor and recipient are completely identical. There is no doubt that immunological rejection reactions against these organs are clearly reduced in terms of their incidence and intensity, and can be better managed therapeutically, if there is the greatest possible conformity for the HLA antigens. For the transplantation of liver, lung, and pancreas, it has not been possible to prove clinically, up to the present time, that the immunological rejection risk can be reduced by means of HLA conformity between patient and donor. However, it was possible to show that preformed, donor-specific anti-HLA antibodies, or those formed after transplantation, result in humoral rejection, going as far as loss of function of the transplanted organ, in every case.

The detection of anti-HLA antibodies is a central component of pre- and post-transplantation diagnostics, within the framework of the transplantation of blood stem cells and solid organs. Screening for HLA antibodies always takes place before transplantation in the case of all patients for stem cell transplantation and all patients who are on waiting lists for the transplantation of solid organs, and after transplantation, as a function of the clinical progress. In the case of patients for kidney transplantation, this screening is actually conducted quarterly, in accordance with the guidelines of the Eurotransplant Foundation, in order to reliably identify HLA antibody-positive patients on the waiting lists. In approximately 20% of the patients, HLA antibodies are found that must be specified in additional studies, for characterization of non-transplantable HLA antigens. The methods used for screening and for specification are based on cytotoxicity techniques with vital lymphocytes or ELISA techniques with natural HLA molecules obtained from cell lines. As a result, identification of the antibody specificities is possible only with great restrictions, because of the co-expression of all HLA loci as well as the coupling non-equilibrium between alleles of the various HLA loci.

In the case of highly immunized patients, specification of the antibodies is not possible in almost any of the cases, using these techniques. But specifically in these cases, identification of acceptable mismatches, i.e. of HLA antigens against which no antibodies have been formed, is particularly significant, since this frequently offers the only chance of finding a compatible donor.

These restrictions can be solved by using recombinant human MHC proteins, rhMHC, by means of the use of a defined antigen per reaction, as ELISA, or by means of the flow-through cytometric technique. By using all the rhMHC antigens per reaction, an effective antibody screening system can furthermore be established. Using rhMHC, the problem of the limited availability of rear HLA alleles for screening purposes can furthermore be solved. Recombinant human MHCs have recently been available as prokaryotic and eukaryotic proteins, which can be produced according to the methods described below.

The long-term goal of an allogenic blood stem cell transplantation SCT is, for one thing, establishing a permanent T cell response against tumor antigens that restrict MHC and, for another thing, formation of tolerance with regard to healthy cells. The potential of allogenic T cells for checking malignant cell growth is known from the close coupling between graft-versus-host (GvH) and graft-versus-leukemia (GvL) reaction, and it was possible to show this impressively by means of the reinduction of remissions by means of T cells after stem cell transplantation. Taking into consideration the results of autologous immune therapy of malignant diseases, with modified tumor cells, tumor lysates, or hybrid cells, on the one hand, and approaches for specific peptide, protein, or DNA vaccination, on the other hand, which have been disappointing up to the present, it can be assumed that successful immune therapy is to be expected in the allogenic system, rather than in the autologous system. The previous data concerning identification of tumor-associated T cell epitopes point in the same direction. On the other hand, the allogenic approach contains the significant disadvantage of rejection and of a severe GvH reaction, which currently stands counter to the general use of stem cells. In order to be able to control the course of an allogenic approach in therapeutically targeted manner, the characterization of MHC ligands as well as their tissue-specific and/or tumor-specific presentation is an absolutely necessary goal. For this purpose, the conclusion of the Human Genome Project was a significant milestone, which makes it increasingly possible to define complete cell-specific expression profiles and which provides the basis for the successor project of genome-wide identification of single nucleotide polymorphisms (SNP). With the continuing advance of the SNP project, constant identification of potential allogenically relevant MHC ligands can be directly expected. A comparable development can be expected in infection biology, with the advancing clarification of microbial genomes and MHC allele-specific peptide-binding motifs.

Virus-specific T cells and the majority of alloreactive T cells recognize MHC molecules in a peptide-dependent manner. Identification and cloning of virus-restrictive and allopeptide-restrictive T lymphocytes is technically very difficult when using conventional methods (cytotoxicity or cytokine expression), and burdened with a very high proportion of non-specific results. Using prokaryotic HLA complexes, it was possible to directly detect peptide-selective CD8⁺ T cells after viral infections, and in the case of autoimmune diseases and tumor patients. Aside from direct visualization, it was possible to show that tetramers are suitable for sorting and cloning allorestrictive CTLs. Parallel with the identification of potential allogenic and microbial MHC ligands, the demand for rhMHC which, equipped with these ligands, can be used for identifying targetable T cell epitopes, will increase. As soon as MHC allele-specific T cell epitopes have been identified, the cascade of diagnostic and therapeutic applications can start. The same holds true for the detection of NK cells, whose receptors represent ligands for MHC proteins. Here, however, it is unclear whether this reaction takes place in peptide-selective manner, or independent of the presented peptide.

Recombinant human MHC proteins can be produced in prokaryotes or eukaryotes. The production in eukaryotes takes place either truncated or complete. The truncated variant is expressed without the transmembranous segment and without the cytoplasmic segment, as in the case of prokaryotic expression. Therefore the recombinant molecule lacks the possibility of anchoring in the cell membrane, so that the protein is secreted by the cells. Such a protein is described in the U.S. patent document 2003/0191286 A1. In the case of this protein, there is the disadvantage that the protein is missing the cytosolic tail, although it belongs to the soluble part of the protein.

In the production of complete recombinant molecules, the protein is expressed in the cell membrane. However, the disadvantage here lies in the fact that no soluble proteins are produced.

The production in prokaryotes takes place as a truncated molecule, whereby the molecule is expressed without the transmembranous segment and without the cytoplasmic segments. In this connection, the individual chains are expressed independently in prokaryotes, and folded into a functional MHC molecule in vitro. In addition to the absence of the cytosolic tail, the disadvantages of this method consist in the fact that the folding effort is very great, the proteins are missing the natural glycolisation, and as a rule, only a single synthetically produced peptide, which is used for folding, is presented.

It is therefore the task of the invention to propose an MHC protein and a method for its production, which is soluble but nevertheless has the same folding, activity, and epitopes accessible for antibodies outside of the membrane as the native protein.

A solution for this task surprisingly provides that the recombinant protein is soluble and not truncated, in other words cut off at the end. Several advantages result from this. Because of the solubility, the yield in the production of the recombinant proteins is higher, for one thing. The proteins do not remain in or on the cell membrane, but rather are secreted. As a result, they are easier to harvest, because they only have to be scooped from the top fraction of the cell cultivation medium. For another thing, the producing cells are not damaged or destroyed, something that is necessary in the case of membrane-positioned proteins, in order to remove them from the membrane. This also improves the yield. Because of their solubility, the proteins are more stable in solution and can be stored better. Since most physiological reactions take place in aqueous solution, it is advantageous if the proteins used as the reagent are also soluble. As a result, they can be used in many different ways. Further advantages of the protein according to the invention result from the fact that it is not truncated. In the case of conventional soluble MHC proteins, the amino acid sequence is completely cut off from the position that represents the beginning of the transmembranous domain. Truncation can be carried out by means of a suitable protease, for example. According to the conventional method, this is done in that the nucleotide sequence is amplified, by means of PCR, only up to the exon that lies before the exon that codes for the membrane part. As a result, this exon is not amplified, but neither are all of the exons that follow it in the sequence, which code for the intracellular domain of the protein. However, this domain can be important for function and conformation. Furthermore, it is the carrier of epitopes for specific antibodies directed against MHC. Therefore it is of significant advantage if a recombinant MHC protein has this domain.

The transmembranous domain of the MHC protein assures its anchoring in the membrane and brings about the lipophilic properties of the protein, which prevent solubility. The MHC protein can now be transformed into a soluble protein because it has no transmembranous domain. This can be achieved, for example in the case of the Class I allele, in that only the allele segments and/or exons that code for the hydrophilic domains, in other words α1, α2, α3, and the tail part, are amplified by means of PCR and suitable primers, while the exon that codes for the transmembranous part is not amplified. If the uninterrupted base sequence amplified in this manner is used for expression of the protein, by means of suitable methods, then the C terminal portion of the protein remains essentially unchanged. The protein therefore has all of the domains, including the cytosolic tail, with the exception of the transmembranous part. Another possibility consists in expressing the entire protein, in principle, including the membrane part, but modifying the latter with regard to its amino acid sequence. For example, one or more codons can be changed within the exon that codes for the membrane part, so that hydrophobic amino acids are exchanged for hydrophilic ones. The replacement of a codon can also have the purpose of functionally changing the conformation of the transmembranous domain, in such a manner that it can no longer fulfill its anchoring function, and therefore the entire protein becomes soluble. For this purpose, the replacement of only one codon and/or one amino acid can already be sufficient.

Because the recombinant MHC protein has a cytosolic tail, it has approximately the same conformation, functional activity, and epitope structure as the native protein.

It is advantageous that the recombinant MHC protein according to the invention has the glycolisation of the native MHC protein. Carbohydrates bound to proteins are essential for the conformation and function of these proteins. The glycolisation is furthermore important for the recognition of MHC protein structures by means of components of the immune system, for example antibodies.

The recombinant MHC protein can be isolated in that it is purified by means of affinity chromatography and/or gel chromatography. A simple method for affinity-chromatographic purification consists of the use of a monoclonal antibody against the tag sequences of the recombinant molecules, e.g. anti-His, anti-V5.

The recombinant MHC protein can be an HLA protein of Class I or Class II. As was described above, recombinant HLA proteins, in other words human MHC proteins, are urgently needed for screening transplant patients.

If the recombinant MHC protein has an endogenic peptide bound to its peptide-binding region, this has a number of advantages. For one thing, the MHC protein can be used for detection of the presented peptide, by means of Edman sequencing and mass spectrometry of the eluted peptide. As a result, peptide-binding motifs of the HLA allele in question can be detected, for one thing, and for another thing, the proteins expressed in the cell, in each instance, which then appear as a peptide in the recombinant HLA molecule, can be identified. For another thing, the detection of peptide-binding motifs can be important for vaccine developments. Furthermore, the detection of peptides from endogenically synthesized proteins of the transfected or transduced cells in tumor cells can yield information concerning relevant tumor antigens. Furthermore, the detection of peptides from endogenically synthesized proteins of the transfected or transduced cells in naturally or experimentally virus-infected cells can yield information concerning relevant virus antigens, which information can be important for vaccine developments.

The recombinant MHC protein described is produced essentially in accordance with the following method:

a) Purification of an MHC allele consisting of gDNA or cDNA or RNA.

b) PCR (polymerase chain reaction) amplification of the MHC allele from exon 1 to exon 4 with two suitable primers, preferably with the start primer AE1S and the end primer AE4AS.

c) PCR amplification of an MHC allele, preferably the allele of the first or second method step, up to the end of the coding sequence that lies after MHC allele in exon 7 or 8, in each instance, in other words from exon 6 to exon 7 or exon 8, with two suitable primers, preferably with the start primer AE6S_FS and the end primer AE8AS_WOS, whereby the start primer, preferably AE6S_FS, contains a 5′ sequence that is complementary to the 3′ end of exon 4, so that a fusion sequence can be produced by way of this sequence, and whereby the end primer, preferably AE8AS_WOS, does not contain a stop codon. As a result, parts of the vector can be taken over 3′-wards from the insert, into the transcriptate, which parts code for a tag marking.

d) Joint PCR amplification of the two allele segments obtained in this manner, as an uninterrupted sequence, by means of two suitable primers, particularly using the start primer from method step b, preferably AE1S, and the end primer from method step c), preferably AE8AS_WOS.

e) Cloning of the amplified uninterrupted sequence into a cloning vector, which is also suitable as an expression vector, e.g. pcDNA3.1, so that a vector-insert construct is formed, such as HLAΔE5pcDNA3.1. Alternatively, a simple cloning vector can also be used, and the cloned insert can later be recloned to produce any desired expression vector. The cloned PCR product does not contain a stop codon, so that parts of the vector 3′-wards from the insert can be taken over into the transcriptate. As a result, sequences that code for a tag marking and come from the vector can be taken over, as well. Alternatively, the tag, which can be selected as desired, can also be contained in the primer AE8AS. In this case, the primer is provided with a stop codon 3′-wards from the tag.

f) Expansion of the plasmid obtained in this manner in suitable prokaryotes, preferably in competent E. coli, and subsequent contamination-free purification of the plasmid, for example with Endofree plasmid purification kit, so that transfectable plasmids are obtained.

g) Transfection of eukaryotic cells or cell lines, e.g. K562 or C1R cell lines, with the plasmid. The transfection can take place using any desired method, e.g. electroporation, lipofection, or calcium chloride transfection. The eukaryotic cells can be any desired cells that can naturally form MHC molecules. Cells that cannot form MHC molecules, for example due to lack of expression of the second chain of the MHC molecule and/or the β2 microglobulin, in the case of HLA Class I, can also be used. In this case, the missing chain can be co-transfected in another plasmid or in the same plasmid as the insert described. In the case of MHC Class II, the second chain, the a chain in the case of HLA Class II, must be co-transfected, since it is also anchored in the membrane. The co-transfected chain can be produced in the same manner as the insert described, or take place by means of truncation of the second chain, so that only the extracellular part of this chain is formed. In the case of transduction, the method of procedure is homologous. The transfected or transduced cells then form the recombinant MHC molecules that are secreted into the surrounding medium, since the recombinant MHC Class I molecules are missing the transmembranous segment that is normally coded by exon 5, and the MHC Class II molecules are missing the transmembranous segment that is normally coded by exon 4. The recombinant MHC molecules can be detected in an ELISA. For this purpose, monoclonal antibodies against MHC, e.g. w6/32 against HLA Class I, or against the tag of the protein, e.g. anti-His or anti-V5, can be used.

h) The cells or cell lines that have been modified by means of gene technology can be cultivated and expanded in cell culture flasks and other cell culture techniques. The secreted recombinant MHC molecules can be continuously harvested from the top fraction. Alternatively, the cell lines produced can also be cultivated in hollow-fiber bioreactors, for example from Biovest (BioVest International, Inc., 8500 Evergreen Blvd. NW, Minneapolis, Minn. 55433, USA), and the medium can be continuously harvested.

i) Harvesting of the recombinant MHC molecules secreted by the cells.

j) The recombinant molecules can be isolated from the harvested medium and purified by means of gel chromatography and affinity chromatography. A simple method for affinity-chromatography purification consists of the use of a monoclonal antibody against the tag sequences of the recombinant molecules (e.g. anti-His, anti-V5).

The two following tables show the genomic organization of HLA Class I genes, in Table 1, and the genomic organization of HLA Class II genes, in Table 2. In this connection, the non-coding introns and the coding exons are shown, in each instance, indicating the length of the base pairs bp and the corresponding amino acid position.

TABLE 1
HLA Class I

| Transcribed | Size | Non-coding | Size | Amino acid |
|---|---|---|---|---|
| region(mRNA) | (bp) | region | (bp) | position |
| 5′ UT | 23 | Promoter | 200 | |
| Exon 1 | 73 | Intron 1 | 130 | −24-−1 |
| Exon 2 | 270 | Intron 2 | 241 | 1-90 |
| Exon 3 | 276 | Intron 3 | 601 | 91-182 |
| Exon 4 | 276 | Intron 4 | 97 | 183-274 |
| Exon 5 | 117 | Intron 5 | 438 | 275-313 |
| Exon 6 | 33 | Intron 6 | 140 | 314-324 |
| Exon 7 | 48 | Intron 7 | 169 | 325-340 |
| Exon 8 | 5 | | | 341 |
| 3′ UT | 405 | | | |
| Total size | 1.5 kb | | 2.0 kb | 341 AS |
| Total size of the | | 3.5 kb | | |
| gene | | | | |
| Total size of the | | | | 341 AS |
| protein | | | | |

TABLE 2
HLA Class II

| Transcribed | Size | Non-coding | Size | Amino acid |
|---|---|---|---|---|
| region(mRNA) | (bp) | region | (bp) | position |
| 5′ UT | 62 | Promotor | >200 | |
| Exon 1 | 100 | Intron 1 | 8500 | −29-+4 |
| Exon 2 | 270 | Intron 2 | 2250-2740 | 5-94 |
| Exon 3 | 282 | Intron 3 | 700 | 95-188 |
| Exon 4 | 111 | Intron 4 | 470 | 189-225 |
| Exon 5 | 24 | Intron 5 | 300-840 | 226-233 |
| Exon 6 | 14 | | | 234-238 |
| 3′ UT | >365 | | | |
| Total size | 1.2 kb | | 12.4-13.5 kb | 238 AA |

| | | |
|---|---|---|
| Total size of the | 13.6-14.7 kb | |
| gene | | |
| Total size of the | | 238 AA |
| protein | | |

The invention will be described as an example, in a preferred embodiment, making reference to a drawing, whereby additional advantageous details can be derived from the figures of the drawings.

The figures of the drawings show, in detail:

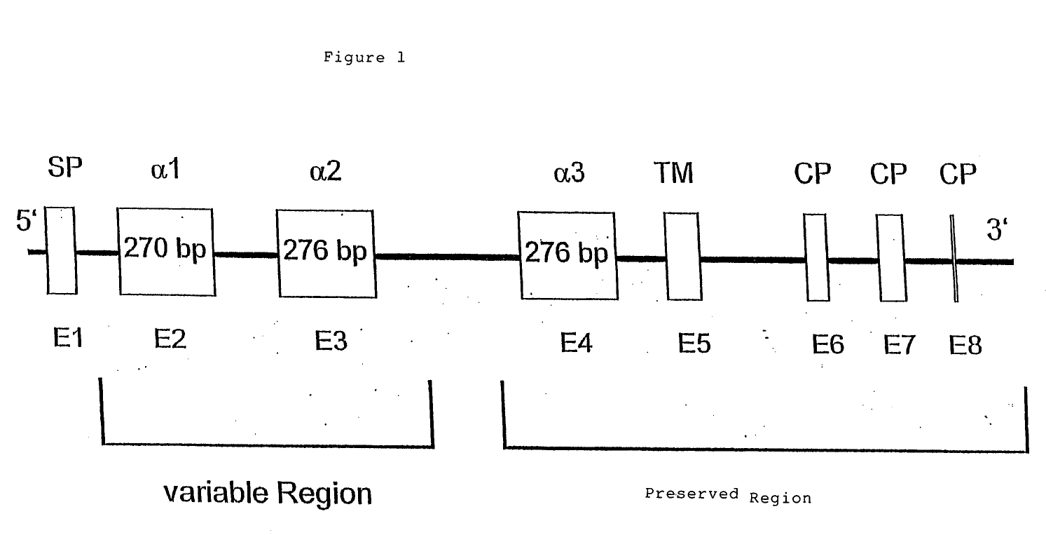

FIG. 1: A schematic representation of the genomic organization of HLA Class I genes;

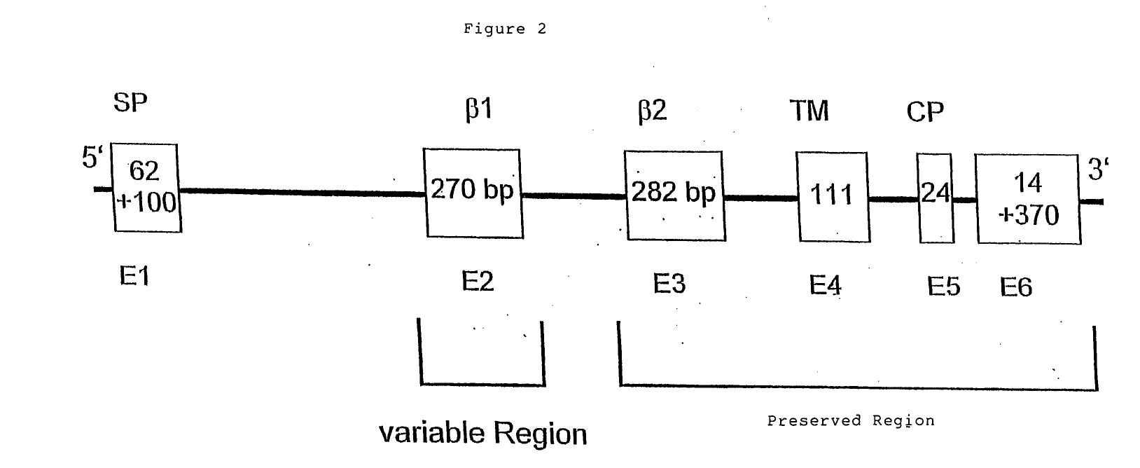

FIG. 2: a schematic representation of the genomic organization as in FIG. 2, but of HLA Class II genes;

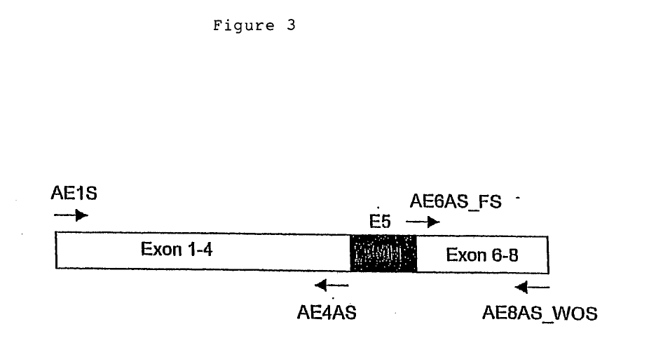

FIG. 3: a schematic representation of the PCR strategy, using the example of HLA-A.

FIG. 1 shows a schematic representation of the genomic organization of HLA Class I genes. In this connection, the nucleotide sequence of the gene is shown on the left 5′ in the direction towards the right 3′. The exons are drawn in as little boxes E1 to E8, whereby the N terminus begins with E1 and the C terminus ends at E8. E1 is the signal peptide SP, which is responsible for transport by way of the membrane after intracellular synthesis of the protein, and E2 and E3 code for the α1 and α2 domains. These are genetically variable and therefore responsible for binding the endogenic peptide. The preserved region is formed by the exons E4 to E8. E4 codes for the α3 domain, ES for the membrane part TM, and the exons E6 to E8 for CP the cytosolic tail.

FIG. 2 shows a schematic representation of the genomic organization as in FIG. 2, but of HLA Class II genes.

FIG. 3 shows a schematic representation of the PCR strategy using the example of HLA-A. Therefore, the essential principle of the invention is being shown. The figure shows the gene sequence as in FIG. 1, but without introns. First, the coding sequence from exon 1 to exon 4 is amplified by means of PCR, with the first start primer AE1S and the first end primer AE4AS. Then, exon 6 to exon 8 of the same allele are amplified with the second start primer AE6S_FS and the second end primer AE8AS_WOS. In this connection, AE6S_FS contains a 5′ sequence that is complementary to the 3′ end of exon 4, so that a fusion sequence can be produced by way of this sequence. The two sequences obtained in this manner, as an uninterrupted sequence, are amplified together with AE1S as the start primer and AE8AS_WOS as the end primer. As a result, the original uninterrupted sequence is formed again, but it is missing exon 5. The almost native protein, without the membrane part, can be produced in soluble form, with this sequence.

In this manner, a soluble recombinant MHC protein is proposed, which is not truncated, and thus in surprising manner has an unchanged C terminus.

1: Recombinant, purified MHC protein, which has essentially the same conformation, functional activity, and binding properties for specific antibodies and antigens as the native MHC protein, wherein it is soluble and not truncated.

2: Recombinant MHC protein according to claim 1, wherein it does not have any transmembranous domains or has a transmembranous domain that is modified with regard to its amino acid sequence.

3: Recombinant MHC protein according to claim 1, wherein it has a cytosolic tail.

4: Recombinant MHC protein according to claim 1, wherein it has the glycolisation of the native MHC protein.

5: Recombinant MHC protein according to claim 1, wherein it is purified by means of affinity chromatography and/or gel chromatography.

6: Recombinant MHC protein according to claim 1, wherein it is an HLA protein of Class I and of Class II.

7: Recombinant MHC protein according to claim 1, wherein it has an endogenic peptide bound to its peptide-binding region.

8: Recombinant MHC protein according to claim 1, wherein it is produced essentially in accordance with the following method:
a) Purification of an MHC allele consisting of gDNA or cDNA or RNA,
b) PCR (polymerase chain reaction) amplification of the MHC allele from exon 1 to exon 4 with two suitable primers, preferably with the start primer AE1S and the end primer AE4AS,
c) PCR amplification of an MHC allele, preferably the allele of the first or second method step, from exon 6 to exon 7 or 8, with two suitable primers, preferably with the start primer AE6S_FS and the end primer AD8AS_WOS, whereby the start primer, preferably AE6S_FS, contains a 5′ sequence that is complementary to the 3′ end of exon 4, and whereby the end primer, preferably AE8AS_WOS, does not contain a stop codon,
d) joint PCR amplification of the two allele segments obtained in this manner, as an uninterrupted sequence, by means of two suitable primers, particularly using the start primer from method step b, preferably AE1S, and the end primer from method step c, preferably AE8AS_WOS,
e) cloning of the amplified uninterrupted sequence into a cloning vector, which is also suitable as an expression vector, or alternatively, recloning of the cloning vector into an expression vector,
f) expansion of the plasmid obtained in this manner in suitable prokaryotes, preferably in competent E. coli, and subsequent purification of the plasmid,
g) transfection or transduction of eukaryotic cells or cell lines with the plasmid obtained in this manner,
h) cultivation and expansion of the cells or cell lines, using suitable methods,
i) harvesting of the recombinant MHC molecules secreted by the cells, and
j) purification of the proteins by means of affinity chromatography and/or gel chromatography.

9: Method for the production of a soluble recombinant protein consisting of at least one membrane-positioned and/or transmembranous domain and one or more domains outside of the membrane, whereby a gene that codes for the protein has an exon that codes for the amino acid sequence of each of the domains, in each instance, wherein the exon(s) adjacent in one direction of the sequence are amplified by means of PCR, by means of the selection of suitable primers, which exons code for domains situated outside of the membrane, and are situated in the said direction ahead of the exon(s) that code for the membrane domain, then the exons that are adjacent in one direction of the sequence are amplified by means of PCR, by means of the selection of suitable primers, which exons code for domains situated outside of the membrane, and are situated in the said direction behind the exon(s) that code for the membrane domain; the sequences amplified in this manner are amplified as uninterrupted sequences, by means of PCR and suitable primers; the uninterrupted sequence is cloned as a cloning vector and/or expression vector and/or expanded in cells; the cells thus modified, producing the protein without the membrane part, and the expressed soluble proteins are harvested and purified by means of conventional or other suitable methods.

10: Use of the MHC protein according to claim 1, wherein it is used for the detection of anti-HLA antibodies, for the detection of T cells or NK cells, for carrying out peptide binding assays, or for the detection of peptides presented by the recombinant protein by means of Edman sequencing and mass spectrometry


SEQUENCE LISTING (15 sequences)

SEQ ID NO: 1 - DNA, 18 bp, Homo sapiens
gagatggccgtcatggcg

SEQ ID NO: 2 - DNA, 20 bp, Homo sapiens
ccatctcagggtgaggggct

SEQ ID NO: 3 - DNA, 40 bp, Homo sapiens
ccctcaccctgagatgggatagaaaaggagggagctactc

SEQ ID NO: 4 - DNA, 22 bp, Homo sapiens
cactttacaagctgtgagagac

SEQ ID NO: 5 - DNA, 1098 bp, Homo sapiens
atggccgtcatggcgccccgaaccctcgtcctgctactctcgggggccctggccctgacccagacctgggcgggctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaatgtgaaggcccagtcacagactgaccgagtggacctggggaccctgcgcggctactacaaccagagcgaggacggttctcacaccatccagaggatgtatggctgcgacgtggggccggacgggcgcttcctccgcgggtaccagcaggacgcttacgacggcaaggattacatcgccctgaacgaggacctgcgctcttggaccgcggcggacatggcggctcagatcacccagcgcaagtgggagacggcccatgaggcggagcagtggagagcctacctggagggccggtgcgtggagtggctccgcagatacctggagaacgggaaggagacgctgcagcgcacggacgcccccaagacgcatatgactcaccacgctgtctctgaccatgaggccaccctgaggtgctgggccctgagcttctaccctgcggagatcacactgacctggcagcgggatggggaggaccagacccaggacacggagctcgtggagaccaggcctgcaggggatgggaccttccagaagtgggcgtctgtggtggtgccttctggacaggagcagagatacacctgccatgtgcagcatgagggtctgcccaagcccctcaccctgagatgggagccgtcttcccagcccaccatccccatcgtgggcatcattgctggcctggttctctttggagctgtgatcgctggagctgtggtcgctgctgtgatgtggaggaggaagagctcagatagaaaaggagggagctactctcaggctgcaagcagtgacagtgcccagggctctgatatgtctctcacagcttgtaaagtgtga

SEQ ID NO: 6 - protein, 365 aa, Homo sapiens [sequence omitted]

SEQ ID NO: 7 - protein, 341 aa, Homo sapiens
GSHSMRYFYTSVSRPGRGEPRFIAVGYVDDTQFVRFDSDAASQRMEPRAPWIEQEGPEYWDRNTRNVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVIAGAVVAAVMWRRKSSDRKGGSYSQAASSDSAQGSDMSLTACKV

SEQ ID NO: 8 - DNA, 1116 bp, Homo sapiens
atggccgtcatggcgccccgaaccctcgtcctgctactctcgggggccctggccctgacccagacctgggcgggctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaatgtgaaggcccagtcacagactgaccgagtggacctggggaccctgcgcggctactacaaccagagcgaggacggttctcacaccatccagaggatgtatggctgcgacgtggggccggacgggcgcttcctccgcgggtaccagcaggacgcttacgacggcaaggattacatcgccctgaacgaggacctgcgctcttggaccgcggcggacatggcggctcagatcacccagcgcaagtgggagacggcccatgaggcggagcagtggagagcctacctggagggccggtgcgtggagtggctccgcagatacctggagaacgggaaggagacgctgcagcgcacggacgcccccaagacgcatatgactcaccacgctgtctctgaccatgaggccaccctgaggtgctgggccctgagcttctaccctgcggagatcacactgacctggcagcgggatggggaggaccagacccaggacacggagctcgtggagaccaggcctgcaggggatgggaccttccagaagtgggcgtctgtggtggtgccttctggacaggagcagagatacacctgccatgtgcagcatgagggtctgcccaagcccctcaccctgagatgggatagaaaaggagggagctactctcaggctgcaagcagtgacagtgcccagggctctgatatgtctctcacagcttgtaaagtgaagggcaattctgcagatatccagcacagtggcggccgctcgagtctagagggcccgcggttcgaaggtaagcctatccctaaccctctcctcggtctcgattctacgcgtaccggtcatcatcaccatcaccattga

SEQ ID NO: 9 - DNA, 135 bp, Homo sapiens
aagggcaattctgcagatatccagcacagtggcggccgctcgagtctagagggcccgcggttcgaaggtaagcctatccctaaccctctcctcggtctcgattctacgcgtaccggtcatcatcaccatcaccat

SEQ ID NO: 10 - DNA, 117 bp, Homo sapiens
agccgtcttcccagcccaccatccccatcgtgggcatcattgctggcctggttctctttggagctgtgatcgctggagctgtggtcgctgctgtgatgtggaggaggaagagctcag

SEQ ID NO: 11 - DNA, 117 bp, Homo sapiens
gagccgtcttcccagcccaccatccccatcgtgggcatcattgctggcctggttctctttggagctgtgatcgctggagctgtggtcgctgctgtgatgtggaggaggaagagctca

SEQ ID NO: 12 - protein, 371 aa, Homo sapiens
MAVMAPRTLVLLLSGALALTQTWAGSHSMRYFYTSVSRPGRGEPRFIAVGYVDDTQFVRFDSDAASQRMEPRAPWIEQEGPEYWDRNTRNVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWDRKGGSYSQAASSDSAQGSDMSLTACKVKGNSADIQHSGGRSSLEGPRFEGKPIPNPLLGLDSTRTGHHHHHH

SEQ ID NO: 13 - protein, 24 aa, Homo sapiens
MAVMAPRTLVLLLSGALALTQTWA

SEQ ID NO: 14 - protein, 45 aa, Homo sapiens
KGNSADIQHSGGRSSLEGPRFEGKPIPNPLLGLDSTRTGHHHHHH

SEQ ID NO: 15 - protein, 347 aa, Homo sapiens
GSHSMRYFYTSVSRPGRGEPRFIAVGYVDDTQFVRFDSDAASQRMEPRAPWIEQEGPEYWDRNTRNVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWDRKGGSYSQAASSDSAQGSDMSLTACKVKGNSADIQHSGGRSSLEGPRFEGKPIPNPLLGLDSTRTGHHHHHH